(19) United States
(12) Patent Application Publication
(10) Pub. No.: US 2026/0083725 A1
(43) Pub. Date: Mar. 26, 2026

(54) SIGMARI LIGAND FOR THE TREATMENT OF PANCREATIC CANCER

(71) Applicants: INSTITUT NATIONAL DE LA SANTÉ ET DE LA RECHERCHE MÉDICALE, Paris (FR); UNIVERSITÉ D'AIX MARSEILLE, Marseille (FR); CENTRE NATIONAL DE LA RECHERCHE SCIENTIFIQUE, Paris (FR); INSTITUT JEAN PAOLI & IRENE CALMETTES, Marseille (FR); UNIVERSITE COTE D'AZUR, Nice (FR); UNIVERSITÉ DE LILLE, Lille (FR); CENTRE HOSPITALIER UNIVERSITAIRE DE LILLE, Lille (FR)

(72) Inventors: Olivier SORIANI, Nice (FR); Mauro-Franck BORGESE, Nice (FR); Raphaël RAPETTI MAUSS, Nice (FR); Richard TOMASINI, Marseille Cedex 09 (FR); Patricia MELNYK, Lille Cedex (FR)

(21) Appl. No.: 19/111,369

(22) PCT Filed: Aug. 9, 2023
(86) PCT No.: PCT/EP2023/072009

§ 371 Date: Mar. 13, 2025

(30) Foreign Application Priority Data
Aug. 10, 2022 (EP) 22306212.6

(51) Int. Cl. A61K 31/4745; A61K 31/7105; A61K 45/06; A61P 35/00; G01N 33/50

(52) U.S. Cl. CPC A61K 31/4745; A61K 31/7105; A61K 45/06; A61P 35/00; G01N 33/5011

(57) ABSTRACT

Here the inventors applied PDAC-derived CAF secretome on pancreatic cancer cells and evaluated Sig-1R implication in stromal cues integration by PCC from signaling transmission to biological outcomes, at the cellular and physiological level. They demonstrated that the loss of Sig-1R in epithelial cells inhibits stromal-induced tumor growth and metastatic process. Thus, the inventors demonstrate that Sig-1R is a key actor of the dialog between stromal and cancer cell compartments The present invention relates to method for the treatment of pancreatic cancer in a patient in need thereof comprising a therapeutically effective amount of a Sig-1R ligand.

### FIELD OF THE INVENTION

The invention is in the field of oncology. More particularly, the invention relates to methods and compositions for the treatment of pancreatic cancer.

### BACKGROUND OF THE INVENTION

Pancreatic Ductal Adenocarcinoma (PDAC) still represents a therapeutic dead-end.

While the overall survival improved by several weeks for patients handling the substantial side effects of combinatory treatments, the global 5 years survival hardly improved. Reported as a typical feature of PDAC as well as one of the main causes of treatment failure, the predominant intra-tumoral microenvironment—or stroma—surrounding pancreatic cancer cells (PCC), is considered as a source of knowledge improvement as well as the niche of new therapeutic targets (1,2). This stromal compartment is composed of immune cells, blood and lymphatic vasculature, nerve fibers, and cancer-associated fibroblasts (CAF) all embedded in an abundant network of extracellular matrix (ECM) proteins. As the main responsible of ECM deposition, and consequently the dense fibrotic reaction observed in PDAC, CAF largely contribute to PDAC aggressiveness. Moreover, by interacting physically with PCC as well as through the secretion of soluble factors (3) or extracellular vesicles (4), CAF promote PCC proliferative, migrative, and invasive potency leading to metastasis formation (5) and exacerbate drugs resistance (6). While CAF were widely described as tumor promoting actors, some studies reported that CAF can restrain PDAC development and PCC abilities.

Delineating CAF subtyping and their respective impact on PDAC and patients' outcome is of crucial importance to improve the therapeutic armamentarium. This situation illustrates the urgent need to understand the physiopathology of PDAC and investigate original drugs targeting the crosstalk between CAF and PCC and its impact on PCC abilities and PDAC evolution.

Primary attempts to target the communication between the tumor microenvironment and PCC revealed that non-specific targeting of tumor microenvironment may enhance tumor development, suggesting a complex interplay between both compartments (7,8). A finer strategy consists in understanding the intercellular crosstalk between CAF and PCC by focusing on specific molecular mechanisms that control this communication (9-11), but the landscape remains incomplete. As transducers of microenvironmental cues, ion channels constitute a family of interface proteins which remains underexplored and offer a promising potency of key inter-cell communication mediators.

Ion channels belong to a family of transmembrane proteins that controls ion fluxes across the plasma membrane, generating electrical signals that tune membrane potential, calcium homeostasis and cell signaling (12-14). A deep remodeling of cell electrical signature accompanies the development of numerous illnesses including cardiopathies, neurodegenerative diseases, inflammation, and cancers. Evidence has been accumulated showing that ion channels strongly contribute to cancer hallmarks such as unrestricted proliferation, evasion of apoptosis, neovascularization, tissue invasion, and formation of metastases (15). Regarding the growing implication of stromal compartment in cancer, deciphering the mechanisms regulating the electrical signature in response to stromal cues seems mandatory to address the intercellular crosstalk in such pathological context. To address this question, the inventors sought to investigate the role of Sigma-1 Receptor (Sig-1R), an ion channel chaperone protein reported in several pathological contexts (16-18), as a mediator of CAF and PCC crosstalk in PDAC.

Here the inventors applied PDAC-derived CAF secretome on pancreatic cancer cells and evaluated Sig-1R implication in stromal cues integration by PCC from signaling transmission to biological outcomes, at the cellular and physiological level. They have found that the secretome obtained from PDAC patients-derived CAF increased cell aggressiveness in vitro and in vivo and identified the SK2 K⁺ channel as mandatory for the transactivation of the Integrin-EGFR-AKT axis. They further confirmed that SK2 acts as a pivotal signaling regulator as being both a direct target of AKT and an amplifier of AKT-downstream transduction. They also determined that Sig-1R is required for the dynamic and spatial formation of the functional membrane complex associating β1-integrin, EGFR, SK2 and AKT in PCC upon CAF stimulation. Significantly, molecular and pharmacological targeting of SK2/Sig-1R inhibited the EGFR-SK2-AKT signaling hub with a consequent reduction of pancreatic cancer cell invasive potency in vitro. Through several in vivo approaches they demonstrated that Sig-1R blockage abrogated CAF-induced metastatic spreading and improved overall survival suggesting that targeting the CAF/PCC crosstalk driven by ion channels represents a potent therapeutic option. Finally, analysis of clinical samples showed higher gene expression of SK2 and Sig-1R in metastases versus primary cancers, and higher expression of SK2 in aggressive molecular subtypes.

### SUMMARY OF THE INVENTION

The invention is in the field of oncology. More particularly, the invention relates to methods and compositions for the treatment of pancreatic cancer. In particular, the present invention is defined by the claims.

### DETAILED DESCRIPTION OF THE INVENTION

Intercellular communication within pancreatic ductal adenocarcinoma (PDAC) tumor microenvironment dramatically contributes to metastatic processes. Yet the underlying mechanisms are poorly understood, resulting in a lack of targeted therapy to counteract stromal-induced cancer cell aggressiveness. Here the inventors report that the K⁺ channel SK2 is stimulated by secreted cues from cancer-associated-fibroblasts (CAF) leading to the activation of an Integrin-EGFR-AKT signaling axis which participates to the acquisition of pro-metastatic features. On another hand they found that Sig-1R is required for the dynamic and spatial formation of the functional membrane complex associating β1-integrin subunit, EGFR, SK2, and AKT upon CAF-induced stimulation of cancer cell. Interestingly, the pharmacological targeting of SK2-dependent signaling using a Sig-1R ligand reduced cancer cell invasive potency in vitro and abrogated CAF-induced metastatic spreading in vivo. Their results establish a new paradigm in which an ion channel shifts the activation-level of a signaling pathway in response to cues from the stroma, opening a new therapeutic window targeting the formation of ion channel-dependent signaling hubs.

The inventor's work highlights an unanticipated role of SK2 channel/Sig-1R chaperone protein complex in mediating intercellular communication between PCC and CAF. They demonstrate that this complex locally amplifies the EGFR-AKT axis upon stimulation by CAF secretome, promoting metastatic spreading and poor survival in pancreatic cancer. They also demonstrated that the loss of Sig-1R in epithelial cells inhibits stromal-induced tumor growth and metastatic process. Thus, the inventors demonstrate that Sig-1R is a key actor of the dialog between stromal and cancer cell compartments, in particular via its modulation.

In a first embodiment, the present invention relates to a method for the treatment of pancreatic cancer in a patient in need thereof comprising a therapeutically effective amount of a Sig-1R ligand.

As used herein, the terms “subject” or “patient” denotes a mammal, such as a rodent, a feline, a canine, and a primate. Particularly, the subject according to the invention is a human. In some embodiments, the patient is an adult. In some embodiments, the subject is more than 15 years old. In some embodiments, the subject is more than 20 years old. In some embodiments, the subject is more than 25 years old. In some embodiments, the subject is more than 30 years old. In some embodiments, the subject is more than 35 years old. In some embodiments, the patient is an elderly.

As used herein, the term “cancer” has its general meaning in the art and refers to a group of diseases involving abnormal cell growth with the potential to invade or spread to other parts of the body. The term “cancer” further encompasses both primary and metastatic cancers.

In some embodiment, the subject has or is susceptible to have pancreatic cancer.

As used herein, the term “pancreatic cancer” or “pancreas cancer” as used herein relates to cancer which is derived from pancreatic cells. In particular, pancreatic cancer included pancreatic adenocarcinoma (e.g., pancreatic ductal adenocarcinoma, PDAC), as well as other tumors of the exocrine pancreas (e.g., serous cystadenomas), acinar cell cancers, intraductal papillary mucinous neoplasms (IPMN) and pancreatic neuroendocrine tumors (such as insulinomas). The cancer may be metastatic cancer. The cancer cells and or tumors that are treated may or may not be resistant to conventional cancer therapy, i.e. the cells in a tumor may exhibit either primary or acquired resistance to conventional cancer therapy and yet they are responsive to (killed by) administration of a Sig-1R ligand.

In some embodiment, the subject has or is susceptible to have pancreatic ductal adenocarcinoma (PDAC).

As used herein, the terms “pancreatic ductal adenocarcinoma” (PDAC) relates to a type of exocrine pancreatic cancer. It develops from cells lining small tubes in the pancreas called ducts. These carry the digestive juices, which contain enzymes, into the main pancreatic duct and then on into the duodenum (first part of the small intestine). PDAC can grow anywhere in the pancreas, though it is most often found in the head of the pancreas. Symptoms can include tummy (abdominal) and back pain, weight loss and changes to bowel habits.

As used herein, the term “stroma” denotes the intra-tumoral microenvironment. Tumors are composed of neoplastic cells and non-neoplastic cells in various ratio depending tumors type and grade. The total amount of non-neoplastic cells composed the stroma, which are mainly cancer-associated fibroblasts (CAFs) and immune cells. As demonstrated in the present invention, SigmaR1 is involved in stroma/tumor cell crosstalk mediated by extracellular vesicles derived from stroma, improving tumor cells abilities, such as tumor cell survival and cancer aggressiveness by enhancing migratory abilities.

As used herein, the terms “cancer-associated fibroblast” (CAF) or “tumor-associated fibroblast” or “carcinogenic-associated fibroblast” or “activated fibroblast” refer to a cell type within the tumor microenvironment that promotes tumorigenic features by initiating the remodelling of the extracellular matrix or by secreting cytokines. CAFs are a complex and abundant cell type within the tumor microenvironment; the number cannot decrease, as they are unable to undergo apoptosis. CAFs have been found to be abundant in a tumor stroma.

Myofibroblasts and fibroblasts make up CAFs. The functions of these CAFs have been known to stimulate angiogenesis, supporting the formation of tumors and thus proliferation of cancer cell and metastasis. CAFs are derived from either normal fibroblasts, pericytes, smooth muscle cells, fibrocytes or mesenchymal stem cells. CAFs then go on to support tumor growth by secreting growth factors such as Vascular Endothelial Growth Factor (VEGF), Platelet Derived Growth Factor (PDGF) and Fibroblast Growth Factor (FGF) and other chemokines to stimulate angiogenesis and thus the growth of a tumor.

As used herein, the term “aggressiveness of a cancer” reflects the capacity of a cancer to lead to the formation of metastasis by improving cancer cell dissemination, cancer cell migration and invasion abilities, modifying their adhesive capacities and favoring pre-metastatic and metastatic niche formation. Metastasis represents the growth of cancer cell in a secondary site/organ following cancer cell dissemination from a primary site/organ. In vitro, invasion and migration abilities can be monitored using a Boyden Chamber or using spheroid cell cultures embedded in matrix, in 3D. The skilled person well-know how to detect metastasis in vivo, as example with an echography, a radiology or a scanner.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

The inventors show a new mechanism by which the PDAC stroma stimulates the formation of metastases. This mechanism involves a pair formed by the SigmaR1 chaperone and the SK2 channel which amplifies the sensitivity of an EGFR/Integrine/PI3K/AKT axis. The expression of SK2 and SigmaR1 is also significantly higher in the most aggressive tumor cells (EMT), and in liver metastases. The inventors demonstrate that the pharmacological targeting of SigmaR1 chaperone with a ligand uncouples the SK2 channel from SigmaR1 and inhibits the signaling and therefore the formation of metastases in vivo.

As used herein, the term “sigma receptor” (σ-receptors) refers to protein cell surface receptors that bind different ligands such as 4-PPBP (4-phenyl-1-(4-phenylbutyl) piperidine), SA 4503 (cutamesine), ditolylguanidine, dimethyltryptamine, and siramesine. There are two subtypes of sigma receptors: sigma-1 receptors (σ1) and sigma-2 receptors (σ2), which are classified as sigma receptors for their pharmacological similarities, even though they are evolutionarily unrelated.

As used herein, the term “sigma-1 receptor” (Sig-1R or sigmaR1) refers to a chaperone protein at the endoplasmic reticulum (ER) that modulates calcium signaling through the IP3 receptor. In humans, the Sig-1R is encoded by the SIGMAR1 gene. The Sig-1R is a transmembrane protein expressed in many different tissue types. It is particularly concentrated in certain regions of the central nervous system. It has been implicated in several phenomena, including cardiovascular function, schizophrenia, clinical depression, the effects of cocaine abuse, and cancer. Sig-1R is an integral membrane protein with 223 amino acids. The Sig-1R protein is represented by the NCBI reference sequence: Q99720 and the SIGMAR1 gene is represented by the NCBI reference sequence: 10280.

As used herein, the term “Sig-1R ligand” refers to a substance that modulates the activity of sigma-1 receptor, especially that binds to and regulates the activity of the sigma-1 receptor. Indeed, the Sig-1R ligands behave as disruptors of the interactions between Sig-1R chaperone and the SK2 channel. Here, the inventors show that the ligand 1(S) inhibits the Sig-1R/SK2 interaction and mimics the effect of molecular silencing of Sig-1R.

In a particular embodiment, the Sig-1R ligand is a peptide, peptidomimetic, small organic molecule, antibody, aptamers, siRNA or antisense oligonucleotide. The term “peptidomimetic” refers to a small protein-like chain designed to mimic a peptide.

In a particular embodiment, the Sig-1R ligand is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In particular the Sig-1R ligand is 1(S) having the following chemical formula:

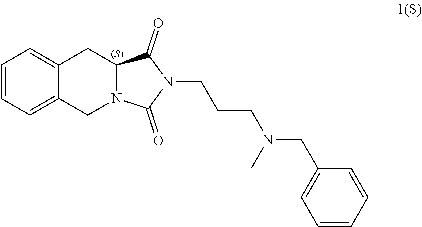

Exemplary of the Sig-1R ligand include also the compounds disclosed in the International Patent Application WO2015193255 and in Donnier-Maréchal M et al. European Journal of Medicinal Chemistry, 2017.

In a particular embodiment, the Sig-1R ligand is siRNA or antisense oligonucleotide.

In a particular embodiment, the Sig-1R ligand is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In some embodiments, the Sig-1R ligand is an antibody. As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388.

In a particular embodiment, the Sig-1R ligand is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular, the Sig-1R ligand is an intrabody having specificity for Sig-1R. As used herein, the term “intrabody” generally refer to an intracellular antibody or antibody fragment.

Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention.

In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb.

In some embodiments, the Sig-1R ligand is a short hairpin RNA (shRNA), a small interfering RNA (siRNA) or an antisense oligonucleotide which modules the expression of Sig-1R. In a particular embodiment, Sig-1R ligand expression is siRNA.

As used herein, the term “short hairpin RNA” (shRNA) relates to a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In order to test the functionality of a putative Sig-1R ligand a test is necessary. For that purpose, to identify Sig-1R ligand an in vitro kinase assay will be used. In brief, recombinant Sig-1R kinase is incubated with a synthetic substrate (SRCtides), ATP as phosphate donor, and increasing concentration of the potential activators. MLR-1023 is used as reference agent, and Staurosporine, (a known nonspecific Sig-1R ligand) as inhibition control.

As used herein the terms “administering” or “administration” refer to the act of injecting or otherwise physically delivering a substance as it exists outside the body (e.g., an ligand of Sig-1R alone or in a combination with a classical cancer treatment) into the subject, such as by, intravenous, intramuscular, enteral, subcutaneous, parenteral, systemic, local, spinal, nasal, topical or epidermal administration (e.g., by injection or infusion). When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The Sig-1R ligand as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected.

These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists.

It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion. Some variation in dosage will necessarily occur depending on the condition of the subject being treated. The person responsible for administration will, in any event, determine the appropriate dose for the individual subject.

A further object of the present invention relates to the combined treatment of Sig-1R ligand with a classical treatment of pancreatic cancer.

In some embodiment, the classical treatment is selected from the group consisting of: radiotherapy, chemotherapy or immunotherapy.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

In some embodiments, the classical treatment consists of administering to the subject a targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs”, “molecularly targeted therapies”, “precision medicines” or similar names.

In some embodiments, the classical treatment consists of radiotherapy. As used herein, the term “radiation therapy” or “radiotherapy” have their general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the X-ray beam, the deeper the X-rays can go into the target tissue. Linear accelerators and betatrons produce X-rays of increasingly greater energy. The use of machines to focus radiation (such as X-rays) on a cancer site is called external beam radiation therapy.

Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In some embodiments, the classical treatment consists of chemotherapy. As used herein, the term “chemotherapy” refers to use of chemotherapeutic agents to treat a subject. As used herein, the term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Angew Chem Int. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino- doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the classical treatment consists of administering to the subject an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively, the immunotherapeutic treatment may consist of administering the subject with an amount of immune cells (T cells, NK, cells, dendritic cells, B cells . . . ).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents. Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors.

Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation).

Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention.

Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin).

In addition to having specific or non-specific targets, immunotherapeutic agents can be active, i.e. stimulate the body's own immune response, or they can be passive, i.e. comprise immune system components that were generated external to the body.

Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

In some embodiments, the immunotherapeutic agent is an immune checkpoint inhibitor.

As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al. 2011. Nature 480:480-489). Examples of stimulatory checkpoint include CD27, CD28, CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumor escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, also knows as Indoleamine 2,3-dioxygenase, is a tryptophan catabolic enzyme and a related immune-inhibitory enzyme. Another important molecule is TDO, tryptophan 2,3-dioxygenase.

IDO is known to suppress T and NK cells, generate and activate Tregs and myeloid-derived suppressor cells, and promote tumor angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyltryptophan (IMT), β-(3-benzofuranyl)-alanine, β-(3-benzo(b)thienyl)-alanine), 6-nitrotryptophan, 6-fluorotryptophan, 4-methyltryptophan, 5-methyltryptophan, 6-methyltryptophan, 5-methoxytryptophan, 5-hydroxytryptophan, indole-3-carbinol, 3,3′-diindolylmethane, epigallocatechin gallate, 5-bromo-4-chloro-indoxyl-1,3-diacetate, 9-vinylcarbazole, acemetacin, 5-bromotryptophan, 5-bromoindoxyl diacetate, 3-aminonaphtoic acid, pyrrolidine dithiocarbamate, 4-phenylimidazole, a brassinin derivative, a thiohydantoin derivative, a β-carboline derivative or a brassilexin derivative. In a particular embodiment, the IDO inhibitor is selected from 1-methyltryptophan, β-(3-benzofuranyl)-alanine, 6-nitro-L-tryptophan, 3-aminonaphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophenyl)-N′-hydroxy-4-{[2-(sulfamoylamino)-ethyl]amino}-1,2,5-oxadiazole-3-carboximidamide:

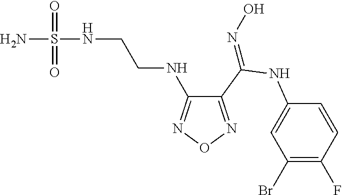 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

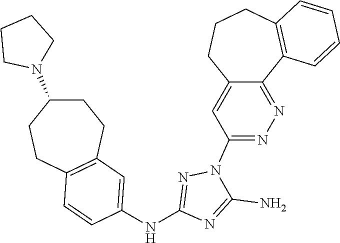 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

Others classical treatment used as anti-cancer agents may be for example cytarabine, anthracyclines, fludarabine, capecitabine, methotrexate, taxol, taxotere, mercaptopurine, thioguanine, hydroxyurea, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. As example, a p38MAPK inhibitor may be SB203580, SB202190, SB202474.

In some embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, MDR inhibitors and Ca2+ ATPase inhibitors.

Additional anti-cancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies. Other additional anti-cancer agent may be selected from, but are not limited to, growth or hematopoietic factors such as erythropoietin and thrombopoietin, and growth factor mimetics thereof.

In the present methods for treating cancer the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dunenhydrinate, diphenidol, dolasetron, meclizme, methallatal, metopimazine, nabilone, oxypemdyl, pipamazine, scopolamine, sulpiride, tetrahydrocannabinols, thiethylperazine, thioproperazine and tropisetron. In a preferred embodiment, the antiemetic agent is granisetron or ondansetron.

In another embodiment, the further therapeutic active agent can be a hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

Typically, the Sig-1R ligand as described above are administered to the subject in the form of a pharmaceutical composition which comprises a pharmaceutically acceptable carrier.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation.

Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and sterile water for injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted considering the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 mL for intramuscular), and between about 1 ng to about 100 mg, e.g. about 50 ng to about 30 mg or more preferably, about 5 mg to about 25 mg, of the ligand of the invention.

A further object of the present invention relates to i) a Sig-1R ligand, and ii) a classical treatment, as a combined preparation for simultaneous, separate or sequential use in the treatment of a cancer.

As used herein, the term “simultaneous use” denotes the use of a Sig-1R ligand and at least one anti-cancer agent occurring at the same time.

As used herein, the term “separate use” denotes the use of a Sig-1R ligand and at least one anti-cancer agent not occurring at the same time.

As used herein, the term “sequential use” denotes the use of a Sig-1R ligand and at least one anti-cancer agent occurring by following an order.

A further object of the present invention relates to a method of screening a drug suitable for the treatment of pancreatic cancer comprising i) providing a test compound and ii) determining the ability of said test compound to module the expression or activity of Sig-1R.

Any biological assay well known in the art could be suitable for determining the ability of the test compound to modulate the activity of Sig-1R. Such assay is briefly described above.

In particular, the effect triggered by the test compound is determined relative to that of the reference compound, in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of modulating the activity of Sig-1R, as determined using in vitro methods described herein, are likely to exhibit similar ligand capacity in applications in vivo. Typically, the test compound is selected from the group consisting of peptides, peptidomimetics, small organic molecules (such as 1(S)), aptamers or nucleic acids.

For example, the test compound according to the invention may be selected from a library of compounds previously synthesised, or a library of compounds for which the structure is determined in a database, or from a library of compounds that have been synthesised de novo.

In some embodiments, the test compound may be selected form small organic molecules.

The invention will be further illustrated by the following figures and examples. However, these examples and figures should not be interpreted in any way as limiting the scope of the present invention.

### FIGURES

FIG. 1: Sig-1R ligand 1(S) inhibits the SK2/Sig-1R complex activity and reduces CAF secretome-induced aggressiveness. A) ISK2 current was recorded in PANC-1 cells exposed overnight to CAF-CM (CM in red n=7) or CM+1(S) ligand 1 μM (Green; n=8). The upper left insert shows the scatter plot representation of the data dispersion and the median (** P<0,01). B) In situ proximity ligation assay analysis of the SK2/AKT interaction in PANC-1 cells Sh RD, treated overnight with control medium, CAF-CM or CAF-CM+1(S) ligand (1 μM). The panel represents the number of interactions per cell in each condition, n=3 experiments * p<0.05; ns: not significant). Scale bar, 10 μm. C) Graphical scheme describing the KIC mice treatment workflow. KIC mice were injected at 5 weeks with vehicle or 1(S) ligand treatments, once a week until each animal reached clinical endpoint. D) Kaplan Meier survival of KIC mice treated with vehicle (n=8) or 1(S) ligand (n=8) from 5 weeks old. Log-Rank (Mantel-Cox) test. E) Graphical scheme describing the KIC mice treatment workflow. KIC mice were injected at 5 weeks with vehicle or 1(S) ligand treatment, once a week until the animals were euthanized at 9 weeks old. F) Graphs showing pancreas weight at end-point of 9 weeks for KIC mice treated with vehicle (n=7) or 1(S) ligand (n=8). G) Graphical scheme describing the KIC mice treatment workflow. KIC mice were injected at 8 weeks with vehicle or 1(S) ligand treatment, once a week until each animal reached clinical endpoint. H) Kaplan Meier survival of KIC mice treated with vehicle (n=8) or 1(S) ligand (n=8) from 8 weeks old. Log-Rank (Mantel-Cox) test. I) Graphs showing the percentage of pan CK labelled cells positive for α-SMA staining in control (untreated, n=7) and 1(S) ligand-treated mice (1(S) ligand, n=8). *p<0.05; **p<0.01; ***p<0.001. CAF, cancer-associated fibroblasts; ns, not significant; PLA, proximity ligation assay.

### EXAMPLE

### Material & Methods

### Drug Treatment

1(S) compound was synthesized as previously described (28,29) and solubilized just before administration.

### Gemm Studies:

Pdx1-Cre, Ink4afl/fl LSL-KrasG12D (KICpdx1) mice were obtained by crossing of the following strains: Pdx1-Cre, Ink4afl/fl, and LSL-KrasG12D mice provided by D. Melton (Harvard Stem Cell Institute, Cambridge, Massachusetts, USA), R. Depinho (Dana-Farber Cancer Institute, Boston, Massachusetts, USA), and T. Jacks (David H. Koch Institute for Integrative Cancer Research, Cambridge, Massachusetts, USA), respectively. PDAC-bearing 5- and 8-week-old mice were treated once a week with vehicle or 1(S) ligand (0.1 mg/injection/mouse) until experimental endpoint mice reaching sacrificing time or mice reaching physiological endpoints (cachexia criteria, limited movement, drinking or feeding of the mice). No exclusion has to be reported in all experiments as no mice reached a criteria of exclusion such as a fight-related wound or the development of ear inflammation.. To study final tumor volume, mice were euthanized at 9 weeks and tumors were weighted and fixed in 4% (wt/vol) formaldehyde for immunochemistry. For survival analyses, mice were euthanized when they developed ethical clinical end points defined by our institutional guidelines and European animal protection law. For all experiments, sample size was predicted following a Mann-Whitney statistical approach involving power calculation of 0.8 with a FDR of 0.05. A minimum number of 7 mice per group was predicted as able to reach significance. Bonferroni post-hoc test was used for multiples test comparison (two variance with time and/or treatment). Experimental technicians were proceeding with injections while personnel related to mice follow-up worked in a blinded approach. All animal care and experimental procedures were performed in agreement with the Animal Ethics Committee of Marseille then the French ministry of research and innovation under the reference Apafis#16998-2018100814458519.

### Orthotopic Tumors Studies:

All the mice were anesthetized by isoflurane (Vetflurane; Virbac) inhalation in 30% air and 70% O₂. NMRI-Nude mice (Janvier Labs) under anesthesia were injected s.c. with 0.2 mg/kg buprenorphine (Vetergesic; Sogeval) and were administered lidocaine (Xylovet; Ceva) at 3.5 mg/kg by infiltration at the abdominal cavity. A first incision of 8 mm was made at the top left of the abdomen and a second at the peritoneum to reach the spleen and attached pancreas. PANC Sh RD/PANC Sh Sig-1R/PANC Sh SK2 cells alone (500,000 cells) or PANC Sh RD/PANC Sh Sig-1R/PANC Sh SK2 (500,000 cells) plus CAF (1,500,000 cells) contained in 40 μL of PBS were injected into the pancreas. The abdominal musculature of the mouse was then closed with a few braided 4-0 sutures using a cutting needle and the external skin closed with inverted stitches. The mice were euthanized 8 weeks later. The pancreas was removed and weighed and the liver removed and analyzed for macroscopic metastases. These organs were fixed in 4% formaldehyde for immunochemistry. Liver metastases were confirmed and counted per mouse using H&E staining. Orthotopic studies were performed in agreement with the Animal Ethics Committee of Marseille then the French ministry of research and innovation under the reference Apafis#16998-2018100814458519.

### Secretome Liquid Chromatography MS/MS Analysis

Ten to 15 mL of secretomes from CAFs (from n=9 tumor patients and from CAF culture immortalized with hTERT (human telomerase reverse transcriptase [CAFhTERT]), were analyzed in biological duplicates by nano-liquid chromatography-MS/MS using an Ultimate 3000 system (Dionex, ThermoFisher Scientific) coupled to an Orbitrap Tribrid Fusion mass spectrometer (Thermo Fisher Scientific). To consider the variabilities arising from secretome biochemical preparation and liquid chromatography MS/MS analyses, normalization across the compared samples was performed by adjusting the medians of the distribution of heavy-labeled protein intensities arising from CAFhTERT (used as an internal standard) for all runs. To do so, a primary CAFhTERT culture was labeled with heavy amino acids (L-arginine and L-lysine) through stable labeling with amino acids in cell culture.

### Clinical Data Analysis

Patient data were accessed through the R2: genomics analysis and visualisation platform (http://r2.amc.n1).

### Cell Culture and CAF Conditioned Media Production

HEK293T, PANC-1 and MiaPaCa-2 were obtained from ATCC cell lines were maintained in Dulbecco's modified Eagle's medium with 10% fetal bovine serum, 100 U/mL peniciline/streptomycin under 5% CO₂, 37° C. in a humidified atmosphere. CAF (Cancer associated fibroblasts) were obtained as previously described (4). Briefly, CAF features were verified by immunofluorescence by a positive α-SMA staining and a negative pan-cytokeratin staining. We also further used a CD45, CD326 (Epcam), Pan-cytokeratin and CD31 FACS sorting to confirm that CAFs cultured are not immune, vascular or tumoral cells. CAFs cultured in DMEM/F12 medium (Gibco; 11330-032), 10% serum (FBS Dutcher), 2 mmol/L L-glutamine (Gibco 25030-024), 1% antibiotic-antimycotic (Gibco 15240-062), and 0.5 mM sodium pyruvate (Sigma S8636) and used between passages 4 and 10. When the monolayer reached approximately 70-80% confluence, they were incubated in DMEM/F12 with 1% FBS, and this conditioned media (CM) was collected every two days and stored at −20° C. Normal Human Fibroblasts (NHF) were isolated from healthy neonatal foreskins and cultured in DMEM, 10% FBS, 2 mmol/l 1-glutamine, 1% antibiotic-antimycotic. Conditioned media was collected as described for CAFs.

### Constructs, Retroviral Production and Transduction

For stable ShRNA cell lines construction: Control neg or Sig-1R-silenced PANC-1 cells were established as previously described (21). Stable SK2 ShRNA PANC-1 cell line was engineered using the following DNA sequence: Sh SK2 for: 5′ TATGGTTCTAATCAGCGTTATCTCGAG ATAACGCTGATTAGAACCATA-3′ (SEQ ID NO: 6). The double stranded DNA sequence was inserted in the mammalian expression vector pSuperRT Puro, a derivative of the pPRIG series. Transduction experiments were performed as previously described (52). Sh Sig-1R res cell line was obtained by transducing C-myc Sig-1R vector in the Sh Sig-1R PANC-1 cell line. For KCNN2 (SK2) gene depletion in PANC-1 cells using CRISPR/Cas9 editing technology: guide RNAs targeting human SK2 were designed as described (53). PANC-1 cells were transfected with pSpCas9(BB)-2A-Puro (PX459) V2.0 plasmid (Addgene plasmid #) carrying gRNAs against human SK2. KCNN2-EX1-pX-FOR: 5′-CACCGCTTAGACACCACGATCTGG-3′ (SEQ ID NO: 1) and KCNN2-EX1-pX-REV: 5′-AAACCCAGATCGTGGTGTCTAAGC-3′ (SEQ ID NO: 2). 48 h after transfection (TransIT-LT1, Mirus), cells were selected in 50 μg/mL puromycin supplemented culture medium. Resistant cells were seeded to a cell per well in 96 wells plate using a Cell sorter ARIA (BD). Single clones were screened for null SK2 associated channel current by patch clamp analysis and western blotting. The protocol for obtaining SK2 mutants is adapted from the QuickChange Site-Directed Mutagenesis kit (Stratagene, La Jolla USA). Forward and reverse primers were obtained from (Sigma-Aldrich). S562A for: 5′-CGG TCC CGG TCC TCG GCC AGG AGG CGG CGG T-3′ (SEQ ID NO: 3), S568A for: 5′-AGG AGG CGG CGG TCC GCT TCC ACA GCA CCA C-3′ (SEQ ID NO: 4), S569A for: 5′-GG CGG CGG TCC TCT GCC ACA GCA CCA C-3′ (SEQ ID NO: 5). Triple mutation was obtained sequentially from single SK2/S562A and double S568A/S569A mutants. Positive clones were detected by DNA sequencing and then sequenced in their entirety to ensure that no polymerase errors had been introduced. HA-myr-AKT is a kind gift from Dr Julie Guillermet-Guibert (CRCT, Toulouse, France).

### Immunochemistry.

Formalin-fixed, paraffin-embedded human sections (4 μm) were deparaffinized in xylene and rehydrated through a graded ethanol series. An antigen retrieval step (Dako) was performed before quenching of endogenous peroxidase activity (3% [vol/vol]H₂O₂). Tissue sections were then incubated with primary antibody (SK2, 1:100, ab9945, Abcam; Sig-1R, 1:100, sc-22948, Santa Cruz Biotechnology) and immunoreactivities were visualized using the Vectastain ABC kit (PK-4005; Vector Laboratories) according to the manufacturers' protocol. Peroxidase activity was revealed using the liquid diaminobenzidine substrate chromogen system (Dako; K3468). Counterstaining with Mayer hematoxylin was followed by a bluing step in 0.1% sodium bicarbonate buffer, before final dehydration, clearance, and mounting of the sections.

### Immunofluorescence.

Formalin-fixed, paraffin-embedded human sections (4 μm) were deparaffinized in xylene and rehydrated through a graded ethanol series. An antigen retrieval step (10 mM sodium citrate, 0.05% Tween 20, 95° C.) was then performed before tissue sections were preincubated in blocking solution (PBS, 0.1% Triton X-100, 10% [vol/vol] donkey serum) for 1 hour. Tissue sections were incubated in a mixture of 2 primary antibodies against SK2 (1:100, ab99457, Abcam), or Sig-1R (1:100, sc-22948, Santa Cruz Biotechnology) with pAKT (1:25, 9271S, Cell signaling), pan-cytokeratin (panCK, 1:100, ab9377, Abcam), or α-smooth muscle actin (α-SMA, 1:100, ab5694, Abcam) in blocking solution (3% BSA, 0.05% Tween20) overnight at 4° C. After washing in PBS, slides were incubated with a mixture of 2 secondary antibodies in blocking solution (Alexa Fluor 568-conjugated, A11057, or Alexa Fluor 488-conjugated antibody, A21206, 1:500, Invitrogen). Stained tissue sections were mounted using Prolong Gold Antifade reagent with DAPI (Life Technologies) before being sequentially scanned at a ×20 magnification under a fluorescent microscope (Nikon Eclipse 90i) equipped with a CCD camera (Nikon DS-1QM).

### Electrophysiology

Electrophysiological recordings in HEK293-T, PANC-1 and MiaPaCa-2 cells were performed in the whole cell configuration of the patch clamp technique at room temperature. Cells were bathed in a solution containing: 5 mM KCl, 145 mM NaCl, 1 mM MgCl₂, 2 mM CaCl₂, and 10 mM Hepes (pH adjusted to 7.4 with HCl, 300 mosm/L). Soft glass patch electrodes (Brand, Wertheim, Germany) were made on a horizontal pipette puller (P-97, Sutter Instrument Co., Novato, CA) to achieve a final resistance ranging from 3 to 5 MΩ. The internal solution was: 145 mM KCl, 1 mM MgCl₂, 0.87 mM CaCl₂, 1 mM EGTA, 10 mM Hepes, 1 mM ATP (pH adjusted to 7.2 with KOH, 308 mosm/L, pCa 6). Recordings were performed at room temperature in the voltage clamp mode using an PC-controlled EPC 9 patch-clamp amplifier (HEKA, Lambrecht/Pfalz, Germany). Currents were acquired and analyzed with Pulse and Pulsefit software (HEKA). Signal were filtered at 10 kHz and digitalized at 20 kHz. Currents were elicited by voltage ramps protocols (from −120 to +60 mV). Current density was measured at 0 mV. For the electrophysiological study of the SK2 mutations, HEK293-T were transfected with the different constructions and the HA-myr-AKT using calcium phosphate and recorded 24 h after transfection.

### In Situ Proximity Ligation Assay

The DuoLink in situ Proximity Ligation Assay (PLA) (Olink Bioscience, Uppsala, Sweden) was used to detect the interaction between SK2 and different candidates' partner (C-myc Sig-1R, EGFR, AKT, β1 integrin). PANC-1 cells expressing a stable Sh R D, Sh Sig-1R, or Sh SK2 were seeded on poly-Lysine (40 μg/mL) coated microscope slides. Cells were fixed with PFA 4% and permeabilized with triton X100 (0.05%). Cells were immunolabeled with primary antibodies: anti SK2 (1:100) and anti C-myc, (1:100) or anti EGFR (1:100), or anti AKT (1:100), or anti β1 integrin (1:100) for 1 h at 37° C. The secondary antibodies with attached PLA probes were supplied in the Duolink kit. Cellular PLA images were captured using an inverted Zeiss Axio Observer Z1 microscope (Zeiss, Jena, Germany). 6 to 10 fields have been imaged and quantified per conditions.

### Western Blot

Cells were lysed using RIPA lysis buffer containing proteases inhibitor cocktail (complete tablets, mini EDTA-free, EASYpack, Roche) and phosphatases inhibitors (PhosSTOP EASYpack, Roche). Whole-cell lysates were subjected to 8% or 10% SDS/PAGE before proteins being transferred onto a polyvinylidene difluoride (PVDF) membrane (Thermo Fisher Scientific). Membranes were probed with the primary antibody overnight at 4° C. Reactive proteins were developed with HRP conjugated secondary antibody and visualized with chemiluminescence according to the manufacturer's protocol (Immobilon® Western, EMD Millipore Corporation) and then exposed to Fusion FX7 Edge (Vilber-Lourmat, France). Details for primary antibodies used are provided in table S. The protein levels were normalised using GAPDH or α-tubulin when appropriate and quantified by image J software (NIH, MD).

### Co-Ip

HEK 293-T cells were transfected using calcium phosphate with WT SK2, HA-myr-AKT or SK2 S562A/S568A/S569A triple mutant. 24 hours later cells were serum starved overnight and then proteins were extracted in IP lysis buffer (co-immunoprecipitation kit; Pierce). Anti-Sk2 (Sigma Aldrich) antibody was immobilized on Aminolink plus coupling resin beads using sodium cyanobrohydride as a cross-linking reagent (75 mM). Samples were added to the SK2-beads complex and incubated overnight at 4° C. on a rotator. The beads were then washed with IP lysis buffer ten times, and then eluted with elution buffer pH 2.8 (Pierce). Samples were subjected to SDS-PAGE and Western blotting assay.

### 3D Matrigel Cell Culture and Filopodia Count

For 3D-culture PANC-1 were cultured in Growth Factor Reduced (GFR) Matrigel (BD Biosciences), briefly PANC-1 cells were trypsinized to a single cell suspension of 5×10³ cells/mL in 1% FBS or CAF-CM completed with 2% GFR Matrigel. Cells in GFR Matrigel were seeded in 8-well coverglass chambers (Nalge Nunc) precoated with 100% GFR Matrigel (for 30 min at 37° C.). Cells were fed every 2 days and grown for 10 days. Samples were fixed in 4% (wt/vol) PFA for 10 min, washed with PBS, and permeabilized with PBS containing 0.5% (vol/vol) Triton X-100 for 10 min. Cells were then washed with PBS, blocked using a solution containing 10% BSA and 10% normal Goat serum, 0.05 Triton X-100 in PBS, and incubated overnight at 4° C. with Alexa Fluor 568 Phalloidin (1/100 in blocking solution). Then samples were exposed to DAPI (1/10 000 in blocking solution) for 10 min, and mounted in moviol mounting medium (Fluka) for further observation. The confocal microscope used to image the samples was a laser scanning confocal microscope LSM710 (Zeiss) with a 63× (NA 1.2 water) objective controlled by ZEN software (2010). Filopodia count were performed using the Image J plugin FiloQuant as described in (54).

### Cell Invasion

Cell invasion assay was performed using CytoSelect™ Cell Invasion Assays from CELL BIOLAB, INC (CBA-11) following the manufacturer's instructions. Briefly, PANC-1 cells stable transduced with a random ShRNA, a Sig-1R ShRNA or the Sk2 KO construct were pretreated with DMEM/F12 or CAF-CM overnight and then seeded at 1×10⁵ cells/well on transwell insert pre-filled with Matrigel. The upper chambers were filled with either DMEM/F12 or CAF-CM and the lower chamber with DMEM/F12 10% FBS. 48 h later cells were incubated for 20 min with CyQuant® fluorescent dye following manufacturer's instruction. Cell invasion was determined by reading fluorescence at 480 nm using a Fluostar Optima microplate reader (BMG Labtech).

### Human Phospho-Kinase Antibody Array

Phospho-antibody array analysis was carried out using the Proteome Profiler Human Phospho-Kinase Array (ARY003B; R&D Systems) according to the manufacturer's instructions Cells were cultured in DMEM/F12 containing 1% FBS or CAF-CM and then lysed with Lysis Buffer 6 (R&D Systems) and agitated for 1 hour at 4° C. Cell lysates were clarified by microcentifugation at 12 000 rpm for 15 minutes, and protein concentration were determined using a Bradford protein assay dye (Bio-Rad). Array membranes were blocked with Array Buffer 1 (R&D Systems) and incubated overnight at 4° C. with 600 g of cell lysate. The membranes were washed to remove unbound proteins and then incubated with biotinylated detection antibodies and streptavidin-HRP. Chemiluminescent detection reagents were applied to detect spot densities. Array images were analyzed using ImageJ software. Array spots were background subtracted and normalized to positive control spots on each membrane to enable comparisons across the different treatment groups. The integrated density of duplicated spots representing each phospho-kinase protein was determined, and data were presented as the fold change compared with the untreated control group. The phospho-antibody array experiment was repeated at least twice, comprising 2 biological and 4 or 6 technical replicates per treatment group.

### EGF Quantification

6.10⁴ PANC-1 cells were seeded in 12-well plates in DMEM 10% FBS. Two days later, at approximately 80% confluence, cultures were rinsed 3 times in PBS and DMEM without FBS was added for 24 hours. Cultures were again rinsed 3 times in PBS and DMEMIF12 1% FBS or conditioned medium from different batches was added. The culture medium was collected 24 hours later, and cells were counted. EGF quantification in culture media was done using kit Quantikine ELISA human EGF (Bio-techne) according to manufacturer instructions. Two different experiments were done with independent cell cultures.

### Fibronectin Quantification

For fibronectin detection, the same culture protocol as for EGF quantification was used except that at 80% confluence, following PBS washing, the culture medium was substituted by DMEM without or with 1% FBS or by conditioned medium from different batches. The culture medium was collected 48 hours later, and fibronectin was quantified by dot blot. Dots of 10 μL serial dilutions of collected culture media were compared to 10 μL DMEM/F12 0% or 1% FBS, conditioned medium and fibronectin concentration ranges from 10 to 0.62 μg/mL The dot blot was probed with anti-fibronectin (AB1949 Millipore) diluted 1/4000 in TBS 0.1% tween 1% unfat milk and secondary anti-rabbit Dako 1/2000. 3 different dot blots were done with 3 independent cell cultures seeded in duplicate in each experiment.

### Gene Expression Analysis of Human PDAC Samples

We gathered clinicopathological and gene expression data of clinical pancreatic samples from 16 publicly available data sets. Data were collected from ArrayExpress, EGA, National Center for Biotechnology Information (NCBI)/Genbank GEO, and TCGA databases and had been generated using DNA microarrays (Affymetrix, Agilent, Illumina) and RNA-seq (Illumina). The pooled data set contained 1,017 samples, including 925 primary PC samples, and 92 metastases. The study was approved by our institutional board. Data analysis required pre-analytic processing as previously published (55). SIGMAR1 expression (codes for Sig-R1) in tumors was measured as discrete value by using the median expression level as cut-off, thus defining two tumor classes thereafter designated “SIGMAR1-high” and “SIGMAR1-low”. KCNN2 expression (codes for SK2) was similarly analyzed. The molecular subtypes of tumors were determined by applying to each sample in each data set separately three different multigene classifiers reported by Bailey (56), Collisson (57) and Moffitt (58). The Baileys' ADEX subtype and the Collisson's Exocrine-like subtype were not considered as their existence is strongly questioned (59). Correlations between the tumor classes based on these two genes and clinicopathological features were analyzed using the t-test or the Fisher's exact test when appropriate: the variables tested included patients' age (≤ vs >60 years) and gender (female vs male), pathological tumor type (ductal vs others) and grade (1 vs 2 vs 3 vs 4), pancreatic anatomic location (head vs body/tail), and American Joint Committee on Cancer (AJCC) stage (1-2 vs 3-4).

### Statistical Analysis

Each experiment was repeated independently at least three times. Results are presented as median with interquartile range unless stated otherwise. For comparison between two quantitative variables, we used the Mann-Whitney test. When more than two variables were compared Kruskal-Wallis tests were used followed by a Dunn's post-test, unless stated otherwise. Analyses were performed using either GraphPad Prism V.8.0.1 Software or R software. A p value <0.05 was considered statistically significant.

### Results

Secreted Cues from Cancer-Associated Fibroblasts (CAF) Stimulate SK2/Sig-1R Complex in Pancreatic Cancer Cells

To determine whether Sig-1R regulates PDAC cells' electrical signature, we recorded ion channel activity in two PCC cell lines (i.e. PANC-1 and MiaPaCa-2) where Sig-1R expression was silenced by Sh RNA. Abrogation of Sig-1R expression led to the inhibition of a potassium conductance exhibiting an inward rectification typical of small conductance calcium-dependent K⁺ channels (SK) (19). Further pharmacological and molecular analyses revealed that the current was suppressed by the SK-channels inhibitor apamin, the SK2-specific inhibitor Dab-7 (Data not shown), and by the silencing of SK2 channel (Data not shown). To investigate the influence of secreted factors from CAF on the Sig-1R-regulated SK2 channel in PCC, we have generated a conditioned media using human primary CAF obtained from PDAC patients (CAF-CM) (Data not shown). We observed that PANC-1 cells incubation with CAF-CM (overnight) increased an apamin- and Dab7-sensitive SK-like current (Data not shown), an effect abolished by the molecular silencing of Sig-1R, and rescued by the expression of C-myc Sig-1R in the sh-cell line (Data not shown). The molecular silencing of SK2 using Sh RNA and Crispr/Cas9 technology suppressed CAF-CM-induced current stimulation in PANC-1 cells (Data not shown). Importantly, CM harvested from Normal Human Fibroblasts (NHF cells) failed to stimulate the current, indicating that the effect is specific to CAF-secretome (Data not shown). Furthermore, boiled CAF-CM had no impact on the current, ruling out any involvement of secreted metabolites (Data not shown). Since Sig-1R modulates ion channel function through tight protein-protein associations (20,21), we asked whether Sig-1R and SK2 could form complexes upon CAF-CM exposure using proximity ligation assay. Because of the lack of Sig-1R antibody that can work in situ we expressed a C-myc Sig-1R in PANC-1 to detect the protein. In PANC-1 cells challenged with CAF-CM, the interaction between SK2 and Sig-1R was significantly increased (Data not shown). Altogether, these results show that CAF-CM triggers the formation of a complex between SK2 and Sig-1R, stimulating SK2-dependent K⁺ current in PCC. Interestingly, immunostaining of human PDAC specimens revealed that Sig-1R and SK2 are effectively expressed in PDAC epithelial cancer cells at the protein level (Data not shown).

### Fibronectinfrom CAF Secretome Activates SK2/Sig-1R Complex Through an Integrin-EGFR-AKT Pathway

To understand how the CAF-CM stimulates SK2 current, we examined the activation state of a set of signaling kinases in PANC-1 cells challenged with CAF-CM using a phosphokinase array. We observed that the most robustly activated kinases were associated to two main signaling clusters: one associated to the EGFR-AKT axis (EGFR, Src, AKT, Wnk1, AMPKa1, mTor) and the other one to integrin signaling (FAK-Src) (Data not shown). We thus evaluated the effects of treatments altering each of these pathways on SK2 activity. Overnight treatment of cells with EGF (25 ng/mL) induced a significant increase in SK2 current, which was dependent on the presence of Sig-1R (Data not shown). By contrast, the EGFR inhibitor Erlotinib and the AKT inhibitor DEBC both suppressed the CAF-CM-induced SK2 current (Data not shown). Together, these data indicate that CAF-CM-induced SK2 stimulation depends on EGFR-associated signaling. Then, given that CAF-CM treatment triggered FAK in the phosphokinase assay, we tested the effect of fibronectin (FN), a FAK-activating element of the tumor microenvironment via the α5β1 integrin in PCC (22,23), and found that FN mimicked the effects of CAF-CM on SK2 current (Data not shown). Accordingly, incubation of cells with a functional anti-β1 integrin antibody abolished the effect of CAF-CM on SK2 channel activity (Data not shown), further demonstrating that CAF-CM-dependent SK2 activation indeed requires integrin/FAK transduction. To confirm the functional links between the different partners at the molecular level, we performed PLA experiments and observed that SK2 forms associations with EGFR, AKT, and the β1 integrin subunit. Moreover, these associations were strongly promoted by CAF-CM treatment and required the presence of Sig-1R, as shown by experiments using PANC-1 cells silenced for the chaperone (Data not shown). These results reveal that SK2 activity is modulated by a signaling hub stimulated by the CAF-CM, stabilized by Sig-1R, which acts as a spatial organizer. We therefore looked for the presence of EGF and FN in CAF-CM. Surprisingly, we were unable to detect EGF, neither in a proteomic analysis of the CAF-CM (Data not shown) nor using a sensible EGF ELISA test (Data not shown). Note that EGF could not be detected in the culture media of PANC-1 cells cultivated with control medium or CAF-CM (Data not shown). In support of this, neither EGF, nor other EGFR ligands (i.e. TGFα, HBEGF, BTC, AREG, EREG and EPGN) could be detected in the proteomic analysis. By contrast, the analysis revealed that FN is one of the most abundant components of CAF secretome (Data not shown), a result confirmed by an immunodot blot assay (Data not shown). Interestingly, the treatment of PANC-1 cells with FN induced the association between AKT and SK2, mimicking the effects of CAF-CM (Data not shown). Finally, these results are globally reinforced by the colocalization between Sig-1R/SK2 and p-AKT obtained by immunohistochemistry from PDAC patient samples (Data not shown). Altogether, these data indicate that components secreted by CAF-CM that stimulate β1-dependent integrins such as FN or Collagen I potentially activate SK2 via the same signaling hub.

### SK2/Sig-1R Complex Drives PCC Aggressiveness in Response to CAF Secretome In Vitro and In Vivo

SK2 and Sig-1R integrate CAF cues in PCC, and consequently stimulates an EGFR-AKT pathway, a main axis involved in tumor development (24), suggesting that an ion channel-driven signaling hub could favour pancreatic cancer cells aggressiveness. To test this hypothesis, we first evaluated the expression of adherent junctions (AJ) markers since loss of AJ coincides with loss of epithelial features and increases invasive and migratory phenotypes (25). Using PCC Sh RD as controls, we confirmed that exposure to CAF-CM provoked a protein levels decrease in E-cadherin, β-catenin, and P-120 catenin—three AJ markers—(Data not shown). As hypothesized, the molecular silencing of either Sig-1R or SK2 abolished the effect of CAF-CM on AJ markers (Data not shown). To further explore this idea, we performed FACS analyses using an epithelial marker (Epcam/CD326) and a mesenchymal marker classically activated following Epithelial-to-Mesenchymal Transition—EMT (CD106)⁶⁰. The addition of CAF-CM on control PCC (ShRD) enhances their EMT phenotype whereas EMT reprogramming is lost in PCC depleted for Sig-1R or SK2 (Data not shown). As a main functional consequence of epithelial markers loss, we then explored invasive potency of PANC-1 cells exposed to CAF-CM. Using a Matrigel invasion assay we confirmed, as suspected, that CAF-CM exposure enhanced significantly PCC invasion abilities (Data not shown). Consistent with epithelial markers data, we further observed that Sig-1R or SK2 deficient PCC did not exhibit CAF-CM driven enhanced invasive potential (Data not shown). Moreover, when grown in 3D condition, CAF-CM increased the formation of protrusions in Sh RD control cells, as shown by F-Actin immunofluoresence (Data not shown) as well as Filopodia density measurement (Data not shown), while such modifications were not observed using Sig-1R or SK2-deficient PCC (Data not shown). Interestingly, Sig1R or SK2 silencing altered neither cell growth, nor cell survival, but strongly decreased cell migration (Data not shown). Interestingly, using a public PDAC patient database through the oncoR2 platform (http://r2.amc.nl; n=130), (26), we revealed that the expression of both Sig-1R and SK2 are negatively correlated to the expression of AJ markers (Data not shown). To reinforce this result, we performed a co-staining between Sig-1R and an epithelial marker (Pan Cytokeratin) or a mesenchymal marker (α-SMA) in PDAC patient samples. Interestingly, Sig-1R and SK2 expression patterns are strictly limited to pan cytokeratin-expressing cells and co-localise with α-SMA (Data not shown). These results suggest that Sig-1R and SK2 are expressed in epithelial cancer cells that have undergone epithelial to mesenchymal transition, a process favored under CAF crosstalk condition, and reported to impact metastasis burden (27). To further challenge this hypothesis, we orthotopically transplanted either Sh RD control, Sh Sig-1R or Sh SK2 PANC-1 cells in presence, or not, of PDAC patients-derived CAF into the pancreas of NMRI-nude mice. As expected, co-injection of Sh RD PCC and CAF revealed an increase in tumor weight (Data not shown) as well as the development of liver metastasis (Data not shown). Interestingly, using Sh Sig-1R or Sh SK2 PCC co-injected with CAF showed no increase in tumoral weight (Data not shown). More importantly we could not observe liver metastasis onset (Data not shown) corroborating our previous hypothesis. Of interest, we controlled the presence of CAFs by the time of euthanasia in co-injected mice by identifying cells only labelled by aSMA staining as a CAF marker, and could validate their presence, consistently with previous publications using CAF/tumor cell orthotopic co-injections (Data not shown). Altogether, these results support the idea that the Sig-1R/SK2 complex constitutes an essential signal transducer for the intercellular communication between CAF and PCC, bolstering PCC aggressiveness and metastasis development.

### a Sig-1R Ligand Inhibits the SK2/Sig-1R Complex Activity and Improves Survival In Vivo

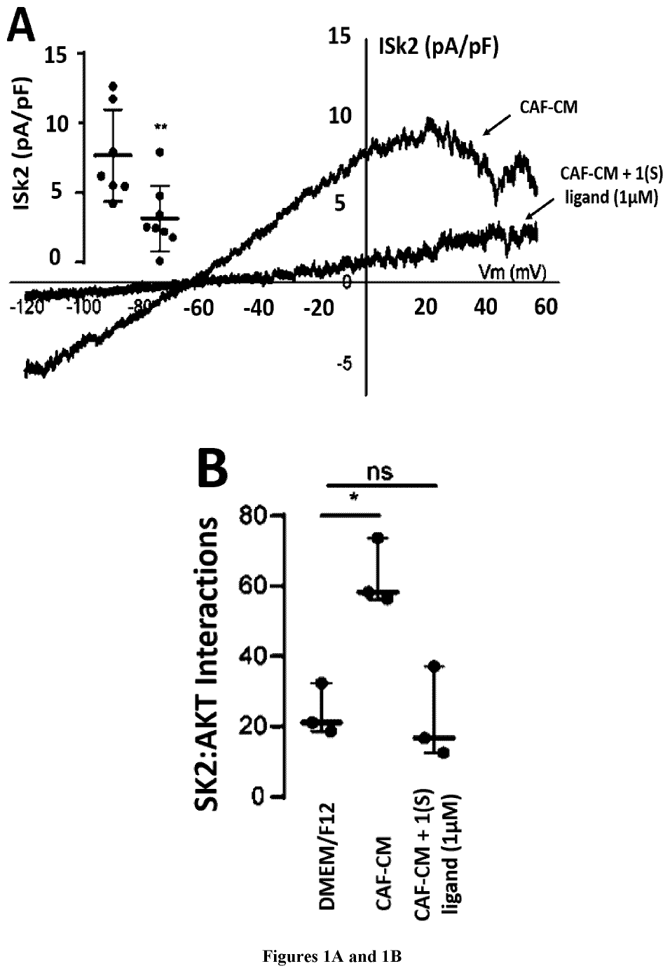

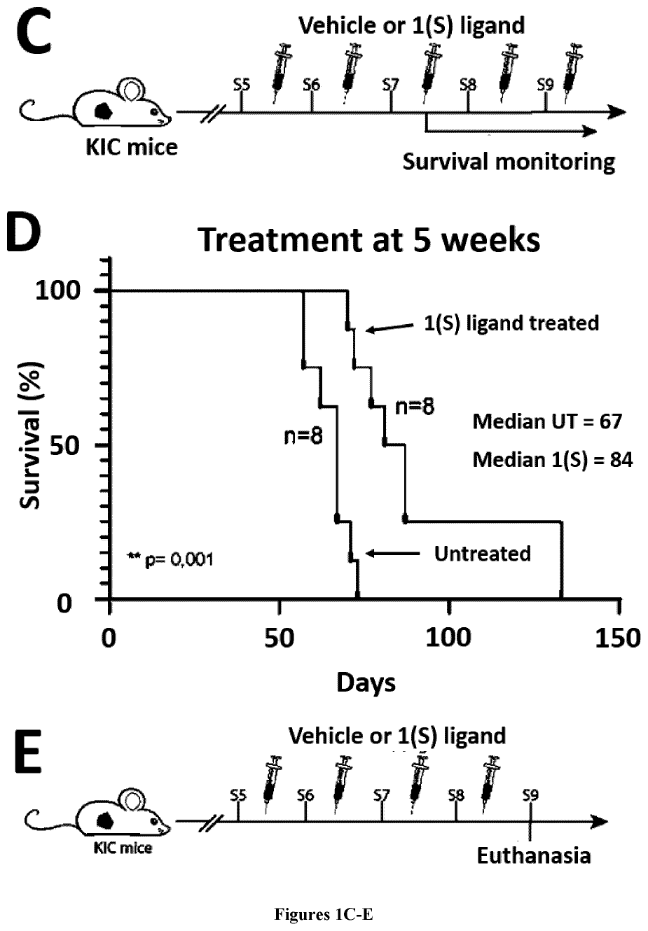

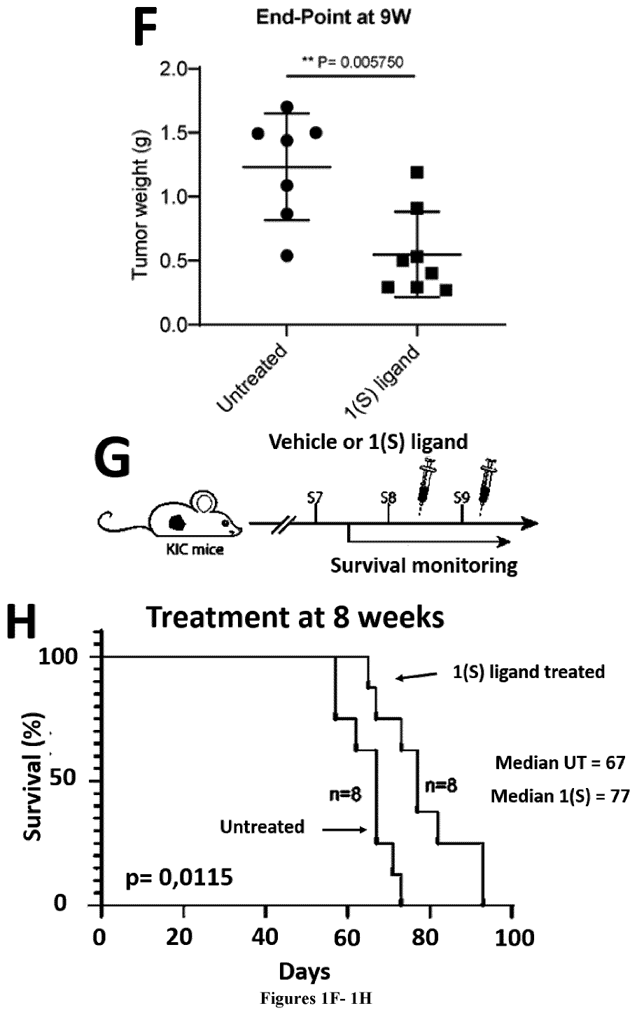

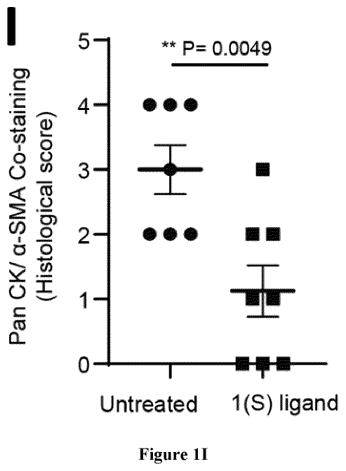

We previously demonstrated that, in pancreatic cancer cells, the SK2/Sig-1R complex acts as a signaling hub which activation drives mesenchymal characteristics favouring metastasis development in vivo. We therefore sought to test whether targeting SK2/Sig-1R in PDAC would be a valuable therapeutic strategy. Sigma ligands are synthetic small molecules that modify specifically the coupling of Sig-1R with its partners including ion channels (17,28). Compound 1(S) is a sigma ligand exhibiting nanomolar affinity for Sig-1R (IC₅₀=3.9 nM), high Sig-2R/Sig-1R selectivity (greater than 120) and very low cytotoxicity (selectivity index, ratio CC₅₀/IC₅₀, greater than 50 000) (29,30). We first validated in vitro that the Sig-1R ligand 1(S) abolished the CAF-CM mediated activation of SK2 and prevented the association of SK2 and AKT as shown by patch-clamp (FIG. 1A) and PLA experiments (FIG. 1B). Proliferation and viability assays, on control Panc-1 cells or cells silenced for Sig1R or SK2, demonstrated that 1(S) treatment does not exhibit any effect on these parameters (Data not shown). We next evaluated the effect of the Sig-1R ligand 1(S) in an endogenous PDAC mice model (KICpdx1 mice) (31). By using a first protocol treating the KICpdx1 mice at 5 weeks old, before the onset of PDAC (FIG. 1C), we observed that Sig-1R ligand 1(S) treatment significantly delayed tumor progression and extended the overall survival. The median survival time of KICpdx1 non-treated animals was 67 days, whereas median survival of KICpdx1-1(S) treated mice was 84 days (FIG. 1D). This data was confirmed on a similar treatment protocol combined to an end-point study at 9 weeks of age (FIG. 1E), representing the median survival of KICpdx1 non-treated mice, validating that Sig-1R ligand 1(S) treatment delayed tumor progression. Indeed, as shown in FIG. 1F, in Sig-1R ligand 1(S) treated KICpdx1 mice, pancreas weight showed a drastic reduction of 69% compared to non-treated ones, decreasing from 1.44 g (±0.42) for KICpdx1non-treated mice to 0.45 g (±0.33) for 1(S) treated KICpdx1 mice. Histological examination of pancreas from 1(S) treated KICpdx1 mice revealed an almost lack of malignant lesions contrasting with pancreas of non-treated KICpdx1 mice exhibiting typical PDAC development (Data not shown). In addition, 1(S)-treated mice exhibited a lower number of EMT-reprogramming epithelial cells (Pan-CK/αSMA co-staining, FIG. 11) and a lower ECM density reflecting a decreased desmoplasia in treated animals (trichrome labelling, Data not shown). To further validate previous data, we have tested the efficiency of the Sig-1R ligand 1(S) treatment on PDAC evolution using an alternative protocol starting the treatment of KICpdx1 mice at 8 weeks of age (FIG. 1G), when PDAC has already developed. Using this experimental design, we confirmed that Sig-1R ligand 1(S) treatment significantly extended the overall survival. Indeed, the median survival time of KICpdx1 non-treated animals was 67 days, whereas median survival of KICpdx1-1(S) treated mice was 77 days (FIG. 1H). Together, these results illustrate that Sig-1R/SK2 inhibition delays PDAC development as well as significantly extends survival, and support that the use of Sig-1R ligand 1(S) is of valuable therapeutic option to treat PDAC.

Sig-1R Expression in PDAC Correlates with Metastatic Phenotype

In order to characterize the expression of Sig-1R in human PDAC, we interrogated publicly available gene expression datasets from 1,017 patients with pancreatic carcinoma (Data not shown). First, we found that SIGMAR1 (coding for Sig-1R: Data not shown), as well as KCNN2 gene (coding for SK2: Data not shown), were more expressed in metastases than in primary tumors, and were more expressed in liver metastases than in non-liver metastases. Second, in an analysis limited to the 925 primary cancers, SIGMAR1 expression, assessed as binary value (cut-off equal to median expression level), was not associated with patients' age and gender, pathological tumor type and grade, and American Joint Committee on Cancer (AJCC) stage (Data not shown). By contrast, high expression was associated with the body/tail pancreatic location (p=3.77E-02). Similar results were observed using SIGMAR1 as continuous value (data not shown). Regarding the molecular subtypes, no correlation existed between SIGMAR1 expression (Data not shown) and the Bailey's classification (pancreatic progenitor, immunogenic, squamous), the Collisson's classification (quasi-mesenchymal, classical), and the Moffitt's tumor classification (basal, classical). By contrast, KCNN2 expression was higher in the squamous Bailey's subtype, the quasi-mesenchymal Collisson's subtype, and the basal Moffitt's tumor subtype (Data not shown). These results revealed higher gene expression of Sig-1R and SK2 in metastases versus primary cancers, and higher expression of SK2 in aggressive molecular subtypes.

### DISCUSSION

The stromal compartment is known to be important in PDAC onset, development and aggressiveness. However, recent studies refined the variety of stromal cellular components such as CAF, and their abilities to potentially support (32-34) or restrain (7, 8, 35) PDAC progression. This knowledge highlighted the necessity to precisely identify and target stromal cues and mechanisms that underlie the pro-tumoral communication between CAF and pancreatic cancer cells. Understanding the specific contribution of the stromal compartment in PDAC physiopathology is mandatory to improve patients care in order to design therapeutic protocols considering the cellular prevalence of stromal actors in PDAC. In the study presented here, we have identified ion channel and chaperone proteins as critical regulators of stromal to tumor cell crosstalk that bolster PDAC development and metastasis. Our results draw a model in which a signaling hub, mainly composed of the potassium channel SK2, the chaperone protein Sig-1R, EGFR and the β1-integrin subunit, is at the interface between the intercellular communication from cancer associated fibroblasts to PDAC cancer cells and the consequent activation of intracellular signaling such as AKT.

Previously, several secreted factors from CAF, within PDAC stroma, such as growth factors (36), cytokines (9), and lipids (11), were shown to mediate paracrine interactions that stimulate PCC aggressive features. Surprisingly, we have been unable to detect EGF in the CAF secretome, although our data show that the CAF-CM mediated activation of SK2 was dependent on EGFR activity. Indeed, this effect was mimicked by EGF treatment and completely abolished by the use of Erlotinib. These results strongly suggest a transactivation mechanism of EGFR, that has been largely reported as integrin related in PDAC (24, 37, 38) and other cancers (39). Moreover, we have shown that CAF-CM exposure triggers the association of SK26, β1 integrin, and EGFR and that this association was dependent on Sig-1R. Thus, reinforcing the idea that CAF-CM stimulates a membrane multiproteic signaling complex rather than only the integrin receptors. We further deepened the CAF-CM-driven transactivation of EGFR and found that fibronectin and collagen I, two major components of CAF-CM activate an Integrin/EGFR/AKT signaling axis that subsequently increases SK2 activity in PCC. The abundant presence of fibronectin and Collagen I has been revealed in stromal PDAC patients and mouse models' samples (40) as well as in the secretome obtained from human primary CAF (41-43). So far, several studies have demonstrated that fibronectin and Collagen I contributes to PCC aggressiveness and reduced survival (44). Altogether, these data support the idea that abundant secretion of FN within the stromal microenvironment, would be a key mediator in the CAF-driven PCC aggressiveness.

We demonstrated here that Sig-1R acts as a keystone in the CAF-induced signaling mechanism suggesting a role for this protein in the formation and dynamics of multiproteic complexes. This hypothesis is further strengthened by our previous study describing that Sig-1R is needed to bring two different ion channels together or ion channel and partner such as integrin (17,18) but also by other studies demonstrating that Sig-1R regulates the functional association between NMDAR and μ-opioid receptor (MOR) (45). Our findings complexify this concept, as Sig-1R is not only necessary to associate several membrane proteins, but is able to organize a whole signaling platform from the membrane reception of stromal signals through β1-integrin-EGFR to the intracellular transduction of the signal via the activation of AKT and its association with SK2. We therefore propose that Sig-1R acts as a spatial organizer of a signaling hub, controlling the dynamics of cell signaling pathway upon stromal driven-extracellular signal activation. Particularly interesting in the context of this study is the observation that SK2 channel activity finely tunes the integrin-EGFR-AKT signaling axis, consequently controlling EMT and metastatic processes in response to CAF influence. Ion channels recently appeared as fundamental molecular actors of cancer cell hallmarks (15). We and others have previously reported that ion channels modulate signaling pathway activity (46-48). Here, we show that the exposure of PCC to CAF-CM stimulates a K⁺ current associated to SK2 channel. On another hand, the silencing of SK2 abolished the activation of Integrin/EGFR/AKT signaling pathway triggered by the CAF secretome as well as PCC aggressiveness, suggesting a central role for this channel in intracellular communication between CAF and PCC. Here we reveal that this channel is a direct target of AKT. Indeed, we identified an AKT-specific motif within the channel sequence containing 2 serine residues that are phosphorylated by CAF-CM treatment, S568 and S569, both being necessary to mediate CAF-CM induced SK2 activity. Interestingly, we found that the AKT specific phosphorylation site in SK2 is highly conserved among species, but is not found in the other members of the SK channel family (i.e. SK1, SK3 and SK4), reinforcing the idea of a functional and specific coupling between SK2 and AKT-dependent pathways. EGFR activation is associated to AKT phosphorylation in PDAC (37) and the EGFR-PI3K-AKT signaling axis is one of the main signaling pathways involved in PDAC progression (49-51). Therefore, we reasoned that the AKT-dependent activation of SK2 could play an important role in the Integrin-EGFR-AKT axis. Our results, showing that SK2 silencing reduced CAF-dependent signalization, suggest that in the absence of a functional channel, PCC remain in a subthreshold state that prohibits the metastatic process upon stimulation by CAF. Accordingly, SK2 inhibition reversed the capacity of CAF to induce EMT, and stabilized cells in an epithelial state. Thus, these data provide arguments to propose that SK2 sets an activation threshold that switches PCC from an epithelial state when SK2 activity is low to a pro-invasive state when channel activity is high. To support this model, the inhibition of SK2 (either directly or by its physical dissociation from the EGFR-Integrin-AKT complex by targeting Sig-1R), decreased CAF-induced cell invasiveness in vitro. Interestingly, silencing of either Sig-1R or SK2 abolished CAF-induced tumor growth and metastasis formation in vivo, correlating with our investigation of human PDAC expression datasets revealing that SK2 and Sig-1R expression are increased in metastasis compared to primary tumors, and even more specifically in liver metastases, in PDAC patients.

Finally, we propose a mechanism for pharmacological intervention, the aim of which is to block the intercellular communication between CAF and PCC in PDAC by targeting the SK2/Sig-1R complex. The results exposed in this report clearly show that the expression of SK2 correlates with the more aggressive subtype of PDAC cancer cells and that the expression of SK2 and Sig-1R is higher in metastatic cells. Reinforced by the description of the molecular mechanism involved, these results strongly support the idea to use SK2/Sig-1R as a pharmacological target to unlock the channel from the signaling hub, impairing the transactivation of downstream oncogenic signaling. In the present study, we demonstrate that PCC treatment with the selective Sig-1R ligand 1(S) abolished CAF-CM-driven physical association between SK2 and AKT, underlying current inhibition. More importantly, pharmacological inhibition of the Sig-1R/SK2 complex was efficient in vivo as mouse models of PDAC treated with 1(S) dramatically delayed tumor development, reduced metastasis onset and extended survival. Because Sig-1R activity is stimulated by stress in diseased tissues⁶¹, the use of Sig-1R ligands should specifically target SK2 in tumors but not in brain or heart. In support of this, a recent study revealed that Sig-1R ligands modulate ion channels in cardiomyocytes from long QT syndrome patients, but not in cardiomyocytes from isogenic controls⁶².

Thus, present data demonstrate that targeted inhibition of Sig-1R is a valuable therapeutic tool to treat PDAC patients. Further evaluation of the Sig-1R/SK2/CAF crosstalk in other solid tumors would open new avenues to consider channel and chaperone proteins targeting as anti-cancer therapeutic options.

In summary, our study elucidates the presence of distinct channel and chaperone proteins driving stromal to tumor cells crosstalk that bolsters pancreatic cancer development and aggressiveness. We have highlighted a channel complex, SK2/Sig-1R, that plays a determinant role in transducing extracellular signals from the tumor microenvironment onto intracellular activation of oncogenic signaling pathways in pancreatic cancer cells. We further determined that under stromal stimulation such signaling hub transactivates EGFR, FAK and AKT without the presence of their classical ligands, allowing a sustained activation of PCC aggressive features. Modulation of the SK2/Sig-1R complex therefore represents an interesting option for experimental therapeutics. We also detailed that the dynamics of the assembly of this signaling hub is under the control of Sig-1R (Data not shown). Although stromal ablation for PDAC treatment remains controversial, our results suggest a potential therapeutic opportunity by targeting a signaling hub through its spatial regulator, Sig-1R, to reduce stromal-induced PDAC aggressiveness. Further prospective studies combining Sig-1R targeting with other therapeutic approaches, as chemotherapeutics agents, are necessary to evaluate their potential as long-term durable anti-PDAC response.

### REFERENCES

1. Kleeff J, Korc M, Apte M, La Vecchia C, Johnson C D, Biankin A V, et al. Pancreatic cancer. Nat Rev Dis Primers 2016; 2:16022 doi 10.1038/nrdp.2016.22.
2. Neesse A, Bauer C A, Öhlund D, Lauth M, Buchholz M, Michl P, et al. Stromal biology and therapy in pancreatic cancer: ready for clinical translation? Gut 2019; 68(1):159-71 doi 10.1136/gutjnl-2018-316451.
3. Schnittert J, Bansal R, Prakash J. Targeting Pancreatic Stellate Cells in Cancer. Trends Cancer 2019; 5(2):128-42 doi 10.1016/j.trecan.2019.01.001.
4. Leca J, Martinez S, Lac S, Nigri J, Secq V, Rubis M, et al. Cancer-associated fibroblast-derived annexin A6+ extracellular vesicles support pancreatic cancer aggressiveness. J Clin Invest 2016; 126(11):4140-56 doi 10.1172/JC187734.
5. Hwang R F, Moore T, Arumugam T, Ramachandran V, Amos K D, Rivera A, et al. Cancer-associated stromal fibroblasts promote pancreatic tumor progression. Cancer Res 2008; 68(3):918-26 doi 10.1 158/0008-5472.CAN-07-5714.
6. Olive K P, Jacobetz M A, Davidson C J, Gopinathan A, McIntyre D, Honess D, et al. Inhibition of Hedgehog signaling enhances delivery of chemotherapy in a mouse model of pancreatic cancer. Science 2009; 324(5933):1457-61 doi 10.1126/science.1171362.
7. Rhim A D, Oberstein P E, Thomas D H, Mirek E T, Palermo C F, Sastra S A, et al. Stromal elements act to restrain, rather than support, pancreatic ductal adenocarcinoma. Cancer Cell 2014; 25(6):735-47 doi 10.1016/j.ccr.2014.04.021.
8. Özdemir B C, Pentcheva-Hoang T, Carstens J L, Zheng X, Wu C C, Simpson T R, et al. Depletion of carcinoma-associated fibroblasts and fibrosis induces immunosuppression and accelerates pancreas cancer with reduced survival. Cancer Cell 2014; 25(6):719-34 doi 10.1016/j.ccr.2014.04.005.
9. Shi Y, Gao W, Lytle N K, Huang P, Yuan X, Dann A M, et al. Targeting LIF-mediated paracrine interaction for pancreatic cancer therapy and monitoring. Nature 2019; 569(7754):131-5 doi 10.1038/s41586-019-1130-6.
10. Zhou L, Husted H, Moore T, Lu M, Deng D, Liu Y, et al. Suppression of stromal-derived Dickkopf-3 (DKK3) inhibits tumor progression and prolongs survival in pancreatic ductal adenocarcinoma. Sci Transl Med 2018; 10(464) doi 10.1126/scitranslmed.aat3487.
11. Auciello F R, Bulusu V, Oon C, Tait-Mulder J, Berry M, Bhattacharyya S, et al. A Stromal Lysolipid-Autotaxin Signaling Axis Promotes Pancreatic Tumor Progression. Cancer Discov 2019; 9(5):617-27 doi 10.1158/2159-8290.CD-18-1212.
12. Huang X, Jan LY. Targeting potassium channels in cancer. J Cell Biol 2014; 206(2):151-62 doi 10.1083/jcb.201404136.
13. Pardo L A, Stuhmer W. The roles of K(+) channels in cancer. Nat Rev Cancer 2014; 14(1):39-48 doi 10.1038/nrc3635.
14. Prevarskaya N, Skryma R, Shuba Y. Ion channels and the hallmarks of cancer. Trends Mol Med 2010; 16(3):107-21 doi 10.1016/j.molmed.2010.01.005.
15. Prevarskaya N, Skryma R, Shuba Y. Ion Channels in Cancer: Are Cancer Hallmarks Oncochannelopathies? Physiol Rev 2018; 98(2):559-621 doi 10.1152/physrev.00044.2016.
16. Soriani O, Kourrich S. The Sigma-1 Receptor: When Adaptive Regulation of Cell Electrical Activity Contributes to Stimulant Addiction and Cancer. Front Neurosci 2019; 13:1186doi 10.3389/fnins.2019.01186.

0 17. Gueguinou M, Crottés D, Chant6me A, Rapetti-Mauss R, Potier-Cartereau M, Clarysse L, et al. The SigmaR1 chaperone drives breast and colorectal cancer cell migration by tuning SK3-dependent Ca(2+) homeostasis. Oncogene 2017 doi 10.1038/onc.2016.501.
18. Crottés D, Rapetti-Mauss R, Alcaraz-Perez F, Tichet M, Gariano G, Martial S, et al. SIGMAR1 Regulates Membrane Electrical Activity in Response to Extracellular Matrix Stimulation to Drive Cancer Cell Invasiveness. Cancer Res 2016; 76(3):607-18 doi 10.1158/0008-5472.CAN-15-1465.
19. Adelman J P, Maylie J, Sah P. Small-conductance Ca2+-activated K+ channels: form and function. Annu Rev Physiol 2012; 74:245-69 doi 10.1146/annurev-physiol-020911-153336.
20. Balasuriya D, D'Sa L, Talker R, Dupuis E, Maurin F, Martin P, et al. A direct interaction between the sigma-1 receptor and the hERG voltage-gated K+ channel revealed by atomic force microscopy and homogeneous time-resolved fluorescence (HTRF®). J Biol Chem 2014; 289(46):32353-63 doi 10.1074/jbc.M114.603506.
21. Crottés D, Martial S, Rapetti-Mauss R, Pisani D F, Loriol C, Pellissier B, et al. Sig1R protein regulates hERG channel expression through a post-translational mechanism in leukemic cells. J Biol Chem 2011; 286(32):27947-58 doi 10.1074/jbc.M111.226738.
22. Jiang H, Hegde S, Knolhoff B L, Zhu Y, Herndon J M, Meyer M A, et al. Targeting focal adhesion kinase renders pancreatic cancers responsive to checkpoint immunotherapy. Nat Med 2016; 22(8):851-60 doi 10.1038/nm.4123.
23. Lee H O, Mullins S R, Franco-Barraza J, Valianou M, Cukierman E, Cheng J D. FAP-overexpressing fibroblasts produce an extracellular matrix that enhances invasive velocity and directionality of pancreatic cancer cells. BMC Cancer 2011; 11:245 doi 10.1186/1471-2407-11-245.
24. Ricono J M, Huang M, Barnes L A, Lau S K, Weis S M, Schlaepfer D D, et al. Specific cross-talk between epidermal growth factor receptor and integrin alphavbeta5 promotes carcinoma cell invasion and metastasis. Cancer Res 2009; 69(4):1383-91 doi 10.1158/0008-5472.CAN-08-3612.
25. Rhim A D, Mirek E T, Aiello N M, Maitra A, Bailey J M, McAllister F, et al. EMT and dissemination precede pancreatic tumor formation. Cell 2012; 148(1-2):349-61 doi 10.1016/j.cell.2011.11.025.
26. Stratford J K, Bentrem D J, Anderson J M, Fan C, Volmar K A, Marron J S, et al. A six-gene signature predicts survival of patients with localized pancreatic ductal adenocarcinoma. PLoS Med 2010; 7(7):e1000307 doi 10.1371/journal.pmed.1000307.
27. Fiori M E, Di Franco S, Villanova L, Bianca P, Stassi G, De Maria R. Cancer-associated fibroblasts as abettors of tumor progression at the crossroads of EMT and therapy resistance. Mol Cancer 2019; 18(1):70 doi 10.1186/s12943-019-0994-2.
28. Kourrich S, Hayashi T, Chuang J Y, Tsai SY, Su T P, Bonci A. Dynamic interaction between sigma-1 receptor and Kv1.2 shapes neuronal and behavioral responses to cocaine. Cell 2013; 152(1-2):236-47 doi 10.1016/j.cell.2012.12.004.
29. Cazenave Gassiot A, Charton J, Girault-Mizzi S, Gilleron P, Debreu-Fontaine M A, Sergheraert C, et al. Synthesis and pharmacological evaluation of Tic-hydantoin derivatives as selective sigmal ligands. Part 2. Bioorg Med Chem Lett 2005; 15(21):4828-32 doi 10.1016/j.bmcl.2005.07.039.
30. Oxombre B, Lee-Chang C, Duhamel A, Toussaint M, Giroux M, Donnier-Marechal M, et al. High-affinity σ1 protein agonist reduces clinical and pathological signs of experimental autoimmune encephalomyelitis. Br J Pharmacol 2015; 172(7):1769-82 doi 10.1111/bph.13037.
31. Collignon A, Silvy F, Robert S, Trad M, Germain S, Nigri J, et al. Dendritic cell-based vaccination: powerful resources of immature dendritic cells against pancreatic adenocarcinoma. Oncoimmunology 2018; 7(12):e1504727 doi 10.1080/2162402X.2018.1504727.
32. Diop-Frimpong B, Chauhan V P, Krane S, Boucher Y, Jain R K. Losartan inhibits collagen I synthesis and improves the distribution and efficacy of nanotherapeutics in tumors. Proc Natl Acad Sci USA 2011; 108(7):2909-14 doi 10.1073/pnas.1018892108.
33. Kaukonen R, Mai A, Georgiadou M, Saari M, De Franceschi N, Betz T, et al. Normal stroma suppresses cancer cell proliferation via mechanosensitive regulation of JMJDla-mediated transcription. Nat Commun 2016; 7:12237 doi 10.1038/ncommsl2237.
34. Provenzano P P, Cuevas C, Chang A E, Goel V K, Von Hoff D D, Hingorani S R. Enzymatic targeting of the stroma ablates physical barriers to treatment of pancreatic ductal adenocarcinoma. Cancer Cell 2012; 21(3):418-29 doi 10.1016/j.ccr.2012.01.007.
35. Chen Y, Kim J, Yang S, Wang H, Wu C J, Sugimoto H, et al. Type I collagen deletion in αSMA. Cancer Cell 2021; 39(4):548-65.e6 doi 10.1016/j.ccell.2021.02.007.
36. Ligorio M, Sil S, Malagon-Lopez J, Nieman LT, Misale S, Di Pilato M, et al. Stromal Microenvironment Shapes the Intratumoral Architecture of Pancreatic Cancer. Cell 2019; 178(1):160-75.e27 doi 10.1016/j.cell.2019.05.012.
37. Kim Y J, Jung K, Baek D S, Hong S S, Kim Y S. Co-targeting of EGF receptor and neuropilin-1 overcomes cetuximab resistance in pancreatic ductal adenocarcinoma with integrin β1-driven Src-Akt bypass signaling. Oncogene 2017; 36(18):2543-52 doi 10.1038/onc.2016.407.
38. Huang M, Anand S, Murphy EA, Desgrosellier J S, Stupack D G, Shattil S J, et al. EGFR-dependent pancreatic carcinoma cell metastasis through Rap1 activation. Oncogene 2012; 31(22):2783-93 doi 10.1038/onc.2011.450.
39. Yoon S O, Shin S, Lipscomb E A. A novel mechanism for integrin-mediated ras activation in breast carcinoma cells: the alpha6beta4 integrin regulates ErbB2 translation and transactivates epidermal growth factor receptor/ErbB2 signaling. Cancer Res 2006; 66(5):2732-9 doi 10.1 158/0008-5472.CAN-05-2941.
40. Tian C, Clauser K R, Öhlund D, Rickelt S, Huang Y, Gupta M, et al. Proteomic analyses of ECM during pancreatic ductal adenocarcinoma progression reveal different contributions by tumor and stromal cells. Proc Natl Acad Sci USA 2019; 116(39):19609-18 doi 10.1073/pnas.1908626116.
41. Amrutkar M, Aasrum M, Verbeke CS, Gladhaug I P. Secretion of fibronectin by human pancreatic stellate cells promotes chemoresistance to gemcitabine in pancreatic cancer cells. BMC Cancer 2019; 19(1):596 doi 10.1186/s12885-019-5803-1.
42. Samain R, Brunel A, Douché T, Fanjul M, Cassant-Sourdy S, Rochotte J, et al. Pharmacologic Normalization of Pancreatic Cancer-Associated Fibroblast Secretome Impairs Prometastatic Cross-Talk With Macrophages. Cell Mol Gastroenterol Hepatol 2021; 11(5):1405-36 doi 10.1016/j.jcmgh.2021.01.008.
43. Dauer P, Zhao X, Gupta V K, Sharma N, Kesh K, Gnamlin P, et al. Inactivation of Cancer-Associated-Fibroblasts Disrupts Oncogenic Signaling in Pancreatic Cancer Cells and Promotes Its Regression. Cancer Res 2018; 78(5):1321-33 doi 10.1158/0008-5472.CAN-17-2320.
44. Jagadeeshan S, Krishnamoorthy Y R, Singhal M, Subramanian A, Mavuluri J, Lakshmi A, et al. Transcriptional regulation of fibronectin by p21-activated kinase-1 modulates pancreatic tumorigenesis. Oncogene 2015; 34(4):455-64 doi 10.1038/onc.2013.576.
45. Rodriguez-Muñoz M, Sanchez-Blizquez P, Herrero-Labrador R, Martinez-Murillo R, Merlos M, Vela J M, et al. The al receptor engages the redox-regulated HINT1 protein to bring opioid analgesia under NMDA receptor negative control. Antioxid Redox Signal 2015; 22(10):799-818 doi 10.1089/ars.2014.5993.
46. Rapetti-Mauss R, Bustos V, Thomas W, McBryan J, Harvey H, Lajczak N, et al. Bidirectional KCNQ1:β-catenin interaction drives colorectal cancer cell differentiation. Proc Natl Acad Sci USA 2017; 114(16):4159-64 doi 10.1073/pnas.1702913114.
47. Zhou Y, Wong C O, Cho K J, van der Hoeven D, Liang H, Thakur D P, et al. SIGNAL TRANSDUCTION. Membrane potential modulates plasma membrane phospholipid dynamics and K-Ras signaling. Science 2015; 349(6250):873-6 doi 10.1 126/science.aaa5619.
48. Ji C D, Wang Y X, Xiang D F, Liu Q, Zhou Z H, Qian F, et al. Kir2.1 Interaction with Stk38 Promotes Invasion and Metastasis of Human Gastric Cancer by Enhancing MEKK2-MEK1/2-ERK1/2 Signaling. Cancer Res 2018; 78(11):3041-53 doi 10.1158/0008-5472.CAN-17-3776.
49. Ardito C M, Grüner B M, Takeuchi K K, Lubeseder-Martellato C, Teichmann N, Mazur P K, et al. EGF receptor is required for KRAS-induced pancreatic tumorigenesis. Cancer Cell 2012; 22(3):304-17 doi 10.1016/j.ccr.2012.07.024.
50. Navas C, Hernández-Porras I, Schuhmacher A J, Sibilia M, Guerra C, Barbacid M. EGF receptor signaling is essential for k-ras oncogene-driven pancreatic ductal adenocarcinoma. Cancer Cell 2012; 22(3):318-30 doi 10.1016/j.ccr.2012.08.001.
51. Chio IIC, Jafarnejad S M, Ponz-Sarvise M, Park Y, Rivera K, Palm W, et al. NRF2 Promotes Tumor Maintenance by Modulating mRNA Translation in Pancreatic Cancer. Cell 2016; 166(4):963-76 doi 10.1016/j.cell.2016.06.056.
52. Martin P, Albagli O, Poggi M C, Boulukos K E, Pognonec P. Development of a new bicistronic retroviral vector with strong IRES activity. BMC Biotechnol 2006; 6:4 doi 10.1186/1472-6750-6-4.
53. Ran F A, Hsu P D, Wright J, Agarwala V, Scott D A, Zhang F. Genome engineering using the CRISPR-Cas9 system. Nat Protoc 2013; 8(11):2281-308 doi 10.1038/nprot.2013.143.
54. Jacquemet G, Paatero I, Carisey A F, Padzik A, Orange J S, Hamidi H, et al. FiloQuant reveals increased filopodia density during breast cancer progression. J Cell Biol 2017; 216(10):3387-403 doi 10.1083/jcb.201704045.
55. Birnbaum D J, Finetti P, Birnbaum D, Mamessier E, Bertucci F. Expression Is a Poor-Prognosis Marker in Pancreatic Adenocarcinoma. J Clin Med 2019; 8(5) doi 10.3390/jcm8050596.
56. Bailey P, Chang D K, Nones K, Johns A L, Patch A M, Gingras M C, et al. Genomic analyses identify molecular subtypes of pancreatic cancer. Nature 2016; 531(7592):47-52 doi 10.1038/nature16965.
57. Collisson E A, Sadanandam A, Olson P, Gibb W J, Truitt M, Gu S, et al. Subtypes of pancreatic ductal adenocarcinoma and their differing responses to therapy. Nat Med 2011; 17(4):500-3 doi 10.1038/nm.2344.
58. Moffitt R A, Marayati R, Flate E L, Volmar K E, Loeza S G, Hoadley K A, et al. Virtual microdissection identifies distinct tumor- and stroma-specific subtypes of pancreatic ductal adenocarcinoma. Nat Genet 2015; 47(10):1168-78 doi 10.1038/ng.3398.
59. Collisson E A, Bailey P, Chang D K, Biankin A V. Molecular subtypes of pancreatic cancer. Nat Rev Gastroenterol Hepatol 2019; 16(4):207-20 doi 10.1038/s41575-019-0109-y.
60. Pastushenko I, Brisebarre A, Sifrim A, et al. Identification of the tumour transition states occurring during EMT. Nature 2018; 556:463-468.
61. Chu U B, Ruoho A E. Biochemical Pharmacology of the Sigma-1 Receptor. Mol Pharmacol 2016; 89:142-53.
62. Song L, Bekdash R, Morikawa K, et al. Sigma non-opioid receptor 1 is a potential therapeutic target for long QT syndrome. Nat Cardiovasc Res 2022; 1:142-156.

1. A method for the treatment of pancreatic cancer in a patient in need thereof comprising administering to the patient a therapeutically effective amount of a Sig-1R ligand.

2. The method according to claim 1, wherein said pancreatic cancer is pancreatic ductal adenocarcinoma (PDAC).

3. The method according to claim 1, wherein the Sig-1R ligand is siRNA.

4. The method according to claim 1, wherein the Sig-1R ligand is a small molecule.

5. The method according to claim 1, wherein said Sig-1R ligand is:

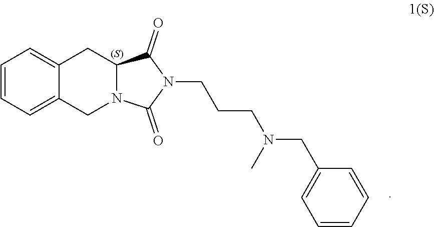

6. The method according to claim 1, wherein the treatment is combined with a classical treatment of pancreatic cancer.

7. The method according to claim 6, wherein the classical treatment is selected from the group consisting of: radiotherapy, chemotherapy and immunotherapy.

8. The method according to claim 6 wherein the method is combined with administration of an immune checkpoint inhibitor.

9. (canceled)

10. A method of screening a drug suitable for the treatment of pancreatic cancer comprising i) providing a test compound and ii) determining the ability of said test compound to modulate expression or activity of Sig-1R.


SEQUENCE LISTING (6 sequences)

SEQ ID NO: 1 - DNA, 24 bp, synthetic construct
  misc_feature 1..24: note = KCNN2-EX1-pX-FOR
  source 1..24: mol_type = other DNA
caccgcttagacaccacgatctgg

SEQ ID NO: 2 - DNA, 24 bp, synthetic construct
  misc_feature 1..24: note = KCNN2-EX1-pX-REV
  source 1..24: mol_type = other DNA
aaacccagatcgtggtgtctaagc

SEQ ID NO: 3 - DNA, 31 bp, synthetic construct
  misc_feature 1..31: note = primer for S562A
  source 1..31: mol_type = other DNA
cggtcccggtcctcggccaggaggcggcggt

SEQ ID NO: 4 - DNA, 31 bp, synthetic construct
  misc_feature 1..31: note = primer for S568A
  source 1..31: mol_type = other DNA
aggaggcggcggtccgcttccacagcaccac

SEQ ID NO: 5 - DNA, 27 bp, synthetic construct
  misc_feature 1..27: note = primer for S569A
  source 1..27: mol_type = other DNA
ggcggcggtcctctgccacagcaccac

SEQ ID NO: 6 - DNA, 48 bp, synthetic construct
  misc_feature 1..48: note = Sh SK2
  source 1..48: mol_type = other DNA
tatggttctaatcagcgttatctcgagataacgctgattagaaccata